(19) United States
(12) Patent Application Publication
(10) Pub. No.: US 2023/0380950 A1
(43) Pub. Date: Nov. 30, 2023

(54) WEARABLE SENSOR APPARATUS FOR MONITORING BODY TEMPERATURE OF PET

(71) Applicants: MAX.K KOREA Inc., Seoul (KR); Max K LLC, Houston, TX (US)

(72) Inventors: Kibun Max KOO, Houston, TX (US); Sang Min AN, Seoul (KR)

(73) Assignees: MAX.K KOREA Inc., Seoul (KR); Max K LLC, Houston, TX (US)

(21) Appl. No.: 18/183,444

(22) Filed: Mar. 14, 2023

(30) Foreign Application Priority Data
May 31, 2022 (KR) 10-2022-0066727

(51) Int. Cl. A61D 13/00; A01K 27/00; A01K 29/00

(52) U.S. Cl. CPC A61D 13/00; A01K 27/001; A01K 29/005

(57) ABSTRACT

The present disclosure relates to a wearable sensor apparatus and method for monitoring a pet body temperature, more particularly, the wearable sensor apparatus being worn on a body of the pet to measure the pet body temperature such that an owner of the pet remotely receives and monitors the measured body temperature, causing no trouble on a skin of the pet even when the wearable sensor apparatus is in contact with the pet skin, and having improved thermal conductivity such that the body temperature can be accurately measured.

### CROSS REFERENCE TO RELATED APPLICATION

This application claims priority to Korea Patent Application No. 10-2022-0066727 filed on May 31, 2022, the content of which is expressly incorporated by reference in its entirety.

### TECHNICAL FIELD

The present disclosure relates to a wearable sensor apparatus for monitoring a body temperature of a pet (i.e., pet body temperature), more particularly, the wearable sensor apparatus being worn on a body of the pet to measure the pet body temperature such that an owner of the pet remotely receives and monitors the measured body temperature, causing no trouble on a skin of the pet even when the wearable sensor apparatus is in contact with the pet skin, and having improved thermal conductivity such that the body temperature can be accurately measured.

### BACKGROUND

In recent years, the demand for pets is greatly increasing for various reasons, such as the improvement of living standards and an increase in emotional demands due to increase of smaller families and single households, and the types of pets are becoming very diverse according to tastes of people, and include not only traditional dogs and cats, but also other mammals, as well as reptiles and amphibians.

There are various methods for managing these pets, and currently provided management methods include simple management functions such as automatically discharging food for a pet left alone when a predetermined time elapses after an owner goes out, or providing the video of the associated pet owner to the pet.

However, although the associated pet could initially respond to simple provision of food or repeated images, the pet does not show any response after learning, so it is not possible to expect an effect for pet management.

In addition, with an existing management method, it is not possible to accurately check health of a pet (e.g., a pet body temperature, etc.), and accordingly, monitoring for checking the pet health in real time is required. That is, it is necessary for an owner to frequently check a pet health from a location right next to the pet or from a remote location through a smartphone and to take appropriate countermeasures as soon as an abnormal condition is found as a result of the checking.

Korean Patent No. 10-1775932 (2017.09.19.) entitled PETS CARE MULTIMODAL SYSTEM AND METHOD THEREFOR, describes big data collected through a multimodal sensor and an iris recognition/biometric sensor for analyzing the psychological status and health status of a companion animal are analyzed, and the physical and emotional situations of the companion animal are identified and managed by deriving a correlation between the behavior of the companion animal and signal characteristics.

Korean Patent Application Publication No. 10-2006-0106804 (2006.10.12.) entitled METHOD FOR MANAGING HEALTH OF ANIMAL BY USING COLLAR FOR ANIMAL AND SYSTEM THEREOF describes a method and system in which health status of an animal can be known by a manager of the animal to prevent emergency situations in advance and respond to the emergency situations quickly, and the manager can continuously monitor the location of an animal that is out of control by using a GPS satellite so that loss of the animal can be prevented.

There is a need and a desire for improved systems and methods for pet monitoring.

### BRIEF SUMMARY OF THE EMBODIMENTS

According to embodiments, a wearable sensor apparatus for monitoring a pet body temperature includes a base constituting a lower part of the wearable sensor apparatus, a cover which is coupled to the base and protects the wearable sensor apparatus and a temperature sensor pocket in which a temperature sensor for measuring the pet body temperature is mounted, wherein the temperature sensor pocket is configured to protrude downward from the base such that the pet body temperature is transmitted to the temperature sensor while the temperature sensor pocket prevents the temperature sensor from directly touching skin of the pet. In addition, the temperature sensor pocket is made of a thermally conductive material so that the wearable sensor apparatus does not cause trouble on the pet skin even when the wearable sensor apparatus is in contact with the pet skin and has improved thermal conductivity such that the body temperature is accurately measured.

Furthermore, the temperature sensor pocket includes an embossing which is exposed to the outside by being coupled to the base and which improves thermal conductivity of the temperature sensor pocket without causing trouble on the pet skin despite contact of the wearable sensor apparatus with the pet skin such that the pet body temperature is accurately measured.

Additionally, the base includes a first coupling hole through which the temperature sensor pocket passes to the outside at which the pet skin is located such that the temperature sensor pocket is coupled to the base, at least one first protruding pin for fixing the temperature sensor pocket, a guide which holds a position of the temperature sensor pocket, at least one screw coupling hole for coupling and fixing the cover to the base, and at least one second protruding pin for fastening the wearable sensor apparatus to a belt.

In addition, the temperature sensor pocket includes a top plate in contact with a bottom surface of the base, at least one second coupling hole which is formed in the top plate and is fitted over and connected to the first protruding pin of the base, a temperature sensor insertion hole depressed into the embossing such that the temperature sensor is inserted into the temperature sensor insertion hole, and a buffer depressed into the top plate for buffering between the embossing and the temperature sensor.

In addition, the buffer is formed by being depressed lower than the top plate such that an end of an FPCB coupled to the temperature sensor is prevented from protruding to the outside of the top plate, the buffer having a circular grooved edge such that the embossing maintains elasticity and strength.

In addition, the wearable sensor apparatus further includes a rechargeable battery, a battery charging module which charges the rechargeable battery, and a communication and processor module which transmits the pet body temperature measured through the temperature sensor to a user terminal or an information collection server, wherein the cover includes a cap having space defined therein such that the battery charging module and the communication and processor module are installed in the space, at least one screw coupling groove formed in a lower part of the cap by being located at a position corresponding to the screw coupling hole of the base such that the screw coupling groove is screwed to the base, at least one fixing groove for fixing the battery charging module and the communication and processor module inside the cap, a charging terminal which is formed on one side of the cap and is connected to a charging cable for charging the battery, a charging cover which is provided outside the charging terminal and is opened during the charging of the battery, a power switch which is formed on one side of the cap and turns on or off power of the wearable sensor apparatus, and a hole for passing light emitted from an LED provided inside the cap to the outside so as to display an operation state of the wearable sensor apparatus.

In addition, in the wearable sensor apparatus, the temperature sensor and the communication and processor module are connected to each other by the FPCB, and the FPCB has a power line and a signal line, and the communication and processor module is provided with an antenna for short range wireless communication and a transceiver for wireless communication.

In addition, the wearable sensor apparatus further includes at least one environmental sensor which measures an ambient temperature, wherein the pet body temperature measured by the temperature sensor and the ambient temperature measured by the at least one environmental sensor are used as independent variables to correct the pet body temperature, and the corrected pet body temperature is transmitted directly to a user terminal, is transmitted to the user terminal via the information collection server, or is transmitted to the information collection server through relay of the user terminal and the user terminal receives the corrected pet body temperature from the information collection server, so that a monitor of the user is capable of monitoring the pet body temperature.

Furthermore, a method of monitoring the pet body temperature according to another embodiment of the present disclosure includes measuring the pet body temperature through the temperature sensor installed in the temperature sensor pocket configured to protrude downward from the base such that the pet body temperature is transmitted to the temperature sensor while the temperature sensor does not directly touch the pet skin, measuring the ambient temperature through the at least one environmental sensor measuring the ambient temperature, and correcting the pet body temperature by adding the pet body temperature measured by the temperature sensor and an atmospheric temperature measured by at least one environmental sensor as independent variables, wherein the corrected pet body temperature is transmitted directly to a user terminal, is transmitted to the user terminal via an information collection server, or is transmitted to the information collection server through relay of the user terminal and the user terminal receives the corrected pet body temperature from the information collection server, so that the user monitor is capable of monitoring the pet body temperature.

Wherein, in the correcting of the pet body temperature, the pet body temperature is corrected by adding difference between the temperature difference and the middle temperature to the measured body temperature after calculating a middle temperature and temperature difference by using a measured body temperature and the ambient temperature, is predicted by applying a least square method to calculate coefficients of a multiple regression equation after deriving a temperature correction table by adding the ambient temperature, the atmospheric temperature, or combination thereof to the measured body temperature, or is predicted by applying the measured body temperature, the ambient temperature, the atmospheric temperature, or combination thereof as input data to an AI learning model after generating the AI learning model by using the measured body temperature, the ambient temperature, the atmospheric temperature, or a combination thereof.

Furthermore, a method of configuring the wearable sensor apparatus for monitoring a pet body temperature according to the embodiment of the present disclosure includes configuring the base which is a base structure of the wearable sensor apparatus, configuring the temperature sensor pocket in which the temperature sensor for measuring the pet body temperature is mounted, configuring the cover which is coupled to the base and protects the wearable sensor apparatus for monitoring the pet body temperature, configuring a printed circuit board including the battery, the battery charging module which charges the battery, and the communication and processor module which transmits the pet body temperature measured by the temperature sensor to a portable terminal of a user through short range wireless communication and corrects a result of the pet body temperature measured by the temperature sensor, and completing the wearable sensor apparatus in such a manner that the printed circuit board is installed in an internal space of the configured cover, opposite sides of the base are connected to the belt at the same time at which the configured temperature sensor pocket is coupled to the configured base, and the cover is coupled to the base, wherein the temperature sensor pocket prevents the temperature sensor from touching directly to the pet skin and is configured to protrude downward from the base such that the pet body temperature is transmitted to the temperature sensor.

As described above, according to the wearable sensor apparatus of the present disclosure, the pet body temperature can be accurately measured through the wearable sensor apparatus which causes no trouble on a pet skin despite the contact of the wearable sensor apparatus with the pet skin and has improved thermal conductivity.

In addition, according to the present disclosure, a user can receive and monitor information about the pet body temperature measured by the wearable sensor apparatus by using his or her portable device, thereby easily checking health of a pet managed by the user and taking immediate countermeasures in the event of an abnormality.

Furthermore, according to the present disclosure, the result of measuring the pet body temperature is corrected and is transmitted to a server in a cloud, and the server in the cloud provides the result of the measured body temperature to a communication terminal of a user, thereby accurately monitoring health of a pet while preventing errors of the measurement of a pet body temperature as much as possible.

Other aspects, features, and techniques will be apparent to one skilled in the relevant art in view of the following detailed description of the embodiments.

### BRIEF DESCRIPTION OF THE DRAWINGS

The features, objects, and advantages of the present disclosure will become more apparent from the detailed description set forth below when taken in conjunction with the drawings in which like reference characters identify correspondingly throughout and wherein:

FIG. 1 is a perspective view of all components comprising a wearable sensor apparatus for monitoring a pet body temperature according to an embodiment of the present disclosure.

FIG. 2 is a side view of the wearable sensor apparatus according to the embodiment of the present disclosure.

FIG. 3 illustrates a top plain view and a bottom view respectively illustrating in detail the structure of a base of the wearable sensor apparatus according to the embodiment of the present disclosure.

FIG. 4 illustrates a top plain view and a side sectional view respectively illustrating in detail the structure of a temperature sensor pocket of the wearable sensor apparatus according to the embodiment of the present disclosure.

FIG. 5 illustrates a side sectional view, a bottom perspective view, and a side view respectively illustrating in detail the structure of a cover of the wearable sensor apparatus according to the embodiment of the present disclosure.

FIG. 6 is a flowchart illustrating in detail the process of configuring the wearable sensor apparatus according to the embodiment of the present disclosure.

FIG. 7 is a view illustrating the concept of correcting a measured value of the pet body temperature in the wearable sensor apparatus according to the present disclosure.

FIGS. 8A, 8B and FIG. 8C are flowcharts illustrating the process of correcting the measured value of the pet body temperature for each embodiment in the wearable sensor apparatus according to the present disclosure.

### DETAILED DESCRIPTION OF THE EXEMPLARY EMBODIMENTS

### Overview and Terminology

Hereinafter, exemplary embodiments of a wearable sensor apparatus for monitoring a pet body temperature of the present disclosure will be described in detail with reference to the accompanying drawings. Like reference numerals in each figure indicate like elements. In addition, specific structural or functional descriptions for the embodiments of the present disclosure are only exemplified for the purpose of describing embodiments according to the present disclosure, and unless defined otherwise, all terms used herein, comprising technical or scientific terms, have the same meaning as commonly understood by those skilled in the art to which the present disclosure belongs. Terms such as those defined in commonly used dictionaries should be interpreted as having meanings consistent with meanings in the context of the related art, and unless explicitly defined in the present specification, the terms should not be interpreted in an ideal or excessively formal meaning.

The present disclosure describes a wearable sensor apparatus in which the wearable sensor apparatus does not cause trouble on the pet skin, even when the wearable sensor apparatus is in contact with a pet skin, and has improved thermal conductivity such that the pet body temperature can be accurately measured, and information about the measured body temperature can be remotely monitored.

On the other hand, according to the present disclosure, the wearable sensor apparatus which causes no trouble on a pet skin despite contact of the wearable sensor apparatus with the pet skin and accurately performs the measurement of the pet body temperature through improved thermal conductivity is attached to the pet body, and the pet body temperature measured through the wearable sensor apparatus is remotely monitored in real time.

The present disclosure has been made to solve problems identified herein, and is intended to propose a wearable sensor apparatus for monitoring a pet body temperature which is worn on a body of a pet to measure the pet body temperature such that an owner of the pet remotely receives and monitors the measured body temperature.

In addition, the present disclosure is intended to propose a wearable sensor apparatus for monitoring a pet body temperature which causes no trouble on a pet skin despite contact of the wearable sensor apparatus with the pet skin and has improved thermal conductivity such that the pet body temperature can be accurately measured.

Furthermore, the present disclosure is intended to propose a wearable sensor apparatus for monitoring the pet body temperature in which the result of the pet body temperature measured by a temperature sensor is corrected according to a multiple regression equation and is transmitted to a server in a cloud, and the server in the cloud provides information about the measured body temperature to a portable device of a user who is an owner of the pet such that the pet body temperature can be monitored.

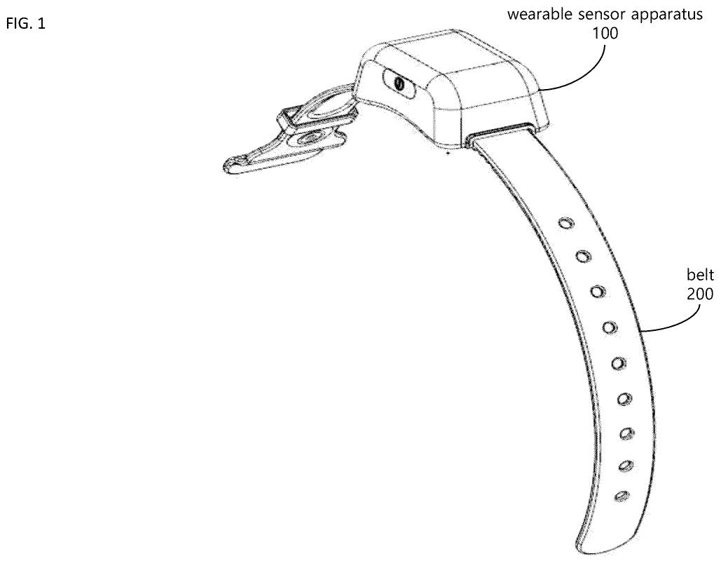

FIG. 1 is a perspective view of all components comprising the wearable sensor apparatus according to an embodiment of the present disclosure.

As illustrated in FIG. 1, according to the present disclosure, the wearable sensor apparatus 100 (hereinafter, referred to as a wearable sensor apparatus) and a belt 200 are provided. In this case, the wearable sensor apparatus 100 is configured to be used by being fastened to a pet body part.

The wearable sensor apparatus 100 is preferably configured in the form of a collar that can be used by being fastened to a neck of the pet, and is used by being fastened to various parts of the pet body, such as the leg or stomach in addition to the neck.

In addition, the wearable sensor apparatus 100 measures the pet body temperature, provides information about the measured body temperature to a remote server or a portable terminal of a user who is the pet owner through wireless communication such as Bluetooth and Wi-Fi such that health of a pet can be monitored in real time. Accordingly, health of the pet raised by the user can be easily checked, and it is possible to take immediate action such as taking the pet to a veterinary hospital in case of abnormality.

Particularly, in the wearable sensor apparatus 100, a part which is in direct contact with the pet skin is made of a material such as eco-friendly silicone and causes no trouble on the pet skin and has improved thermal conductivity such that the pet body temperature can be accurately measured.

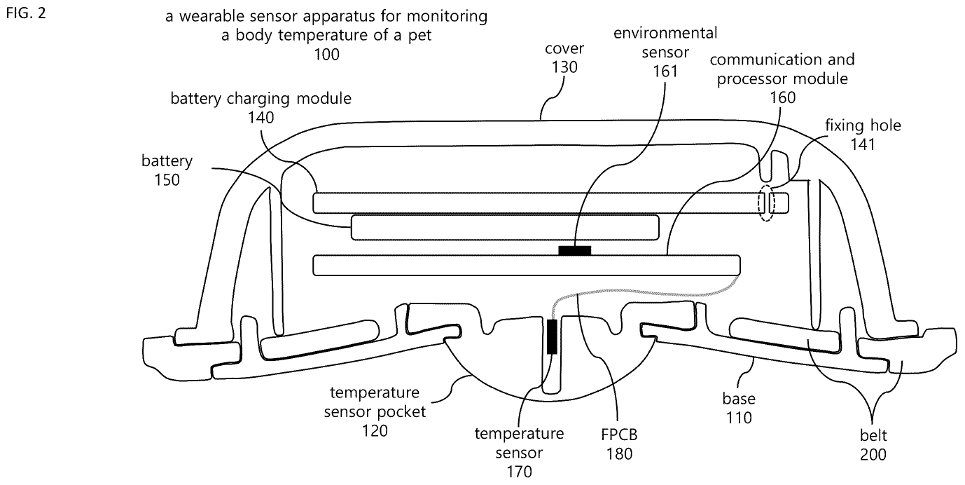

In addition, the wearable sensor apparatus 100 corrects the result of measuring the pet body temperature and transmit the result to the server in the cloud. For example, when the pet body temperature measured through a temperature sensor is outside a preset measurement range (that is, a range of body temperature that can be measured from a pet), the pet body temperature is filtered such that errors of the measurement of the pet body temperature are prevented as much as possible. This will be described in more detail with reference to FIGS. 2, 7, 8A, 8B, and 8C.

FIG. 2 is a side view of the wearable sensor apparatus according to the embodiment of the present disclosure.

As illustrated in FIG. 2, the wearable sensor apparatus 100 comprises a base 110, a temperature sensor pocket 120, a cover 130, a battery charging module 140, a battery 150, a communication and processor module 160, the temperature sensor 170, and a flexible print circuit board (FPCB) 180.

The base 110 is a part constituting the lower part of the wearable sensor apparatus 100 for monitoring the pet body temperature.

In this case, a part of the base 110 with which the pet body is in contact is preferably formed in a streamlined curved shape. This is to enable the wearable sensor apparatus 100 to be fastened naturally to the pet body according to the curve of the pet body.

The temperature sensor pocket 120 is a part in which the temperature sensor 170 for measuring the pet body temperature is mounted, and is coupled to the base 110 toward the lower part of the base 110 from the upper part thereof.

Particularly, the temperature sensor pocket 120 is configured to be coupled to the base 110 by protruding from the lower part of the base 110 such that the pet body temperature can be transmitted to the temperature sensor 170 while the temperature sensor 170 is prevented from directly touching the pet skin.

In addition, the temperature sensor pocket 120 is made of eco-friendly silicone material and prevents trouble on the pet skin despite the contact of the wearable sensor apparatus 100 with the pet skin, and has improved thermal conductivity such that the temperature sensor 170 can accurately measure the pet body temperature.

The cover 130 is formed in the form of a cap having space defined therein and is coupled to the upper part of the base 110, and performs the function of protecting the wearable sensor apparatus 100 for monitoring the pet body temperature.

That is, the printed circuit board comprising the battery charging module 140, the battery 150, and the communication and processor module 160 is fixed and installed in the defined space, and the temperature sensor 170 is inserted into and coupled to the temperature sensor pocket 120 through the FPCB 180 connected to one side of the communication and processor module 160.

The battery charging module 140 controls the battery 150 to be charged with power provided from the outside through a power cable connected to a charging terminal provided on one side of the cover 130.

Reference numeral 141 indicates a fixing hole, and the fixing hole is formed through at least more than one portion of the battery charging module 140 and is a part to which a screw is fastened when fixing the battery charging module 140 in the inner space of the cover 130. In this case, the battery 150 and the communication and processor module 160 are coupled to the lower part of the battery charging module 140, and thus it is sufficient that the fixing hole 141 is formed only in the battery charging module 140.

The battery 150 is filled with power supplied from the outside according to the control of the battery charging module 140, and according to the control of the communication and processor module 160, a charged voltage is supplied to each component necessary for driving the wearable sensor apparatus 100.

The communication and processor module 160 is provided with an antenna for short range wireless communication and a transceiver for wireless communication, and the pet body temperature measured by the temperature sensor 170 is transmitted through the antenna directly to the user portable terminal, or is transmitted through the transceiver for wireless communication to the server in the cloud and the server in the cloud transmits the pet body temperature to the portable terminal of a user who is the pet owner.

In addition, the communication and processor module 160 further comprises an environmental sensor 161. In this case, the environmental sensor 161 performs the function of measuring an ambient temperature of a pet. In addition, the communication and processor module 160 further considers the atmospheric temperature received from a Korea meteorological administration server through a network in addition to the environmental sensor 161 and corrects the pet body temperature.

For example, the communication and processor module 160 corrects and determines the pet body temperature with reference to the pet body temperature measured by the temperature sensor 170, the pet ambient temperature measured by the environmental sensor 161, and the atmospheric temperature provided from the Korea meteorological administration server. In this case, when the corrected and determined pet body temperature is outside a preset range, information about the associated pet body temperature is filtered such that the information is not reflected. The value of an actual coefficient is adaptively determined so that an error is minimized, and the coefficient is calculated through artificial intelligence and a least square method, etc.

Meanwhile, the battery charging module 140, the battery 150, and the communication and processor module 160 are provided on one printed circuit board.

The temperature sensor 170, which is a sensor measuring the pet body temperature, is inserted into and installed in a temperature sensor insertion hole formed in the temperature sensor pocket 120, measures the pet body temperature by heat transfer through the temperature sensor pocket 120 while temperature sensor 170 is not in contact with a pet skin, and provides data for the measured pet body temperature to the communication and processor module 160.

The FPCB 180 has a power line and a signal line, and is located between the temperature sensor 170 and the communication and processor module 160 so as to be connected thereto.

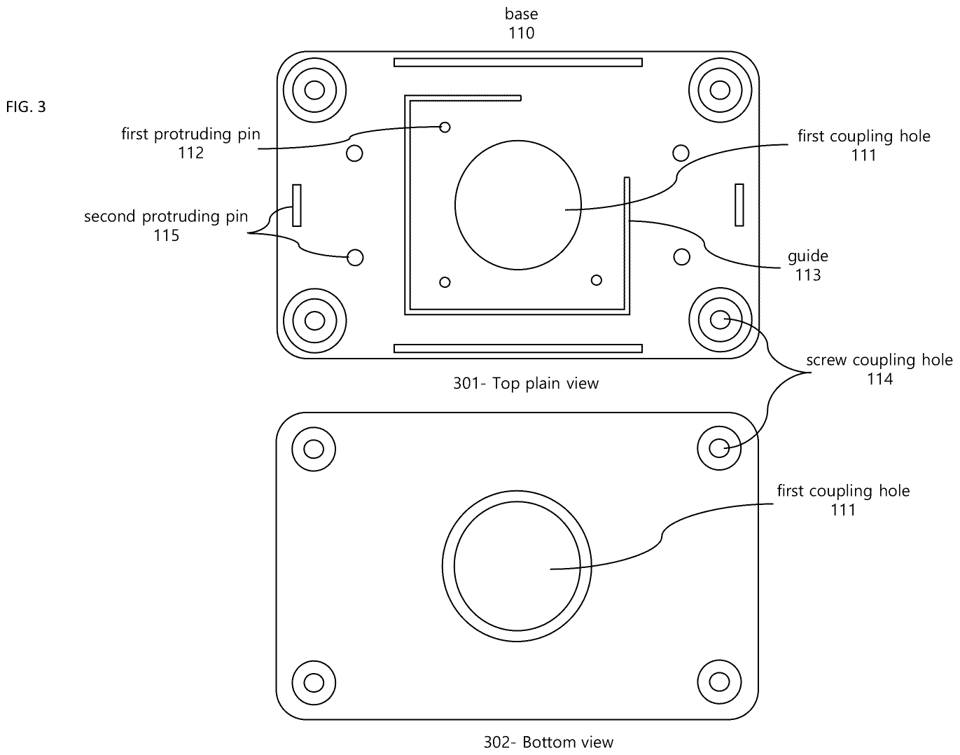

FIG. 3 illustrates a top plain view 301 and bottom view 302 respectively illustrating in detail the structure of a base of the wearable sensor apparatus according to the embodiment of the present disclosure.

As illustrated in FIG. 3, the base 110 includes a first coupling hole 111, at least more than one first protruding pin 112, a guide 113, at least more than one screw coupling hole 114, and at least more than one second protruding pin 115.

The first coupling hole 111 is a part through which the temperature sensor pocket 120 passes to the outside at which the pet skin is located such that the temperature sensor pocket 120 is coupled to the base, and is formed by passing through the center part of the base.

The first protruding pin 112 is a component protruding from a bottom surface of the base 110 for fixing the temperature sensor pocket 120 coupled to the base 110 downward from the upper side of the base 110 through the first coupling hole 111, and comprises at least more than one first protruding pin 112 (e.g., three first coupling holes in top plain view 301 of FIG. 3) formed around the first coupling hole 111.

When the temperature sensor pocket 120 is coupled to the base 110, the guide 113 is a part formed by protruding from the bottom surface to perform the role of holding the coupling position.

That is, when the temperature sensor pocket 120 is fitted over and coupled to each of the first protruding pins 112, the guide 113 performs the role of holding the outer part of the temperature sensor pocket 120 such that the temperature sensor pocket 120 does not move.

The screw coupling hole 114 is a part for coupling and fixing the base 110 to the cover 130, and is preferably formed in each corner of the base 110 by passing therethrough, and screws (other parts that can perform fixing roles can be used) are fastened to the screw coupling holes 114 such that the base 110 and the cover 130 are coupled to each other.

As illustrated in FIG. 3, the second protruding pin 115 is formed to protrude from the bottom surface such that each of the opposite side surfaces of the bottom surface is coupled to the belt 200. In this case, the second protruding pin 115 is formed by protruding in a circular shape or a piece shape.

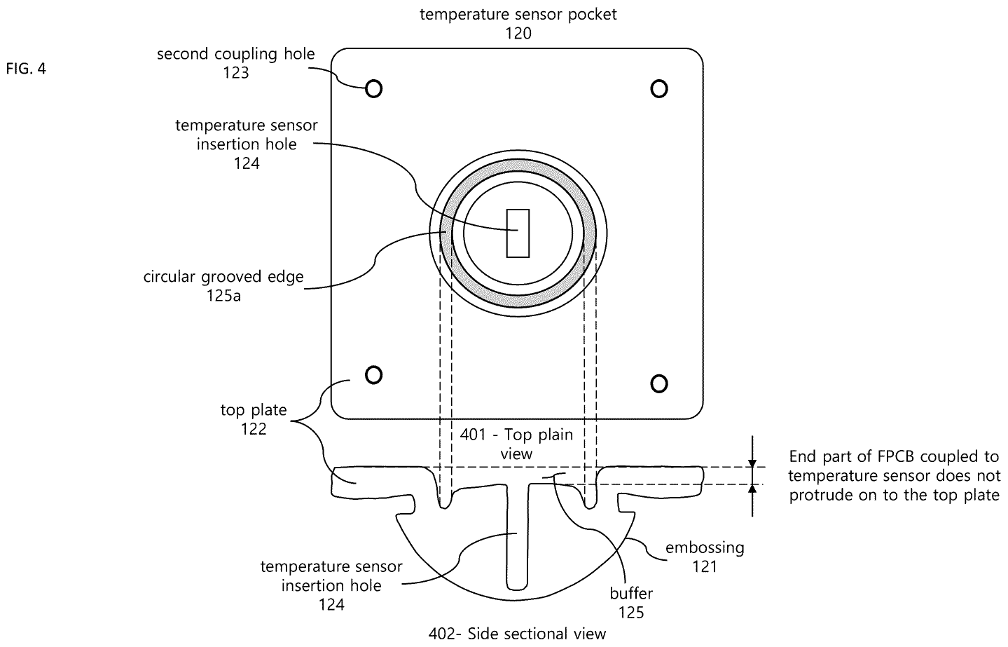

FIG. 4 illustrates a top plain view 401 and a side sectional view 402 respectively illustrating in detail the structure of the temperature sensor pocket of the wearable sensor apparatus according to the embodiment of the present disclosure.

As illustrated in FIG. 4, the temperature sensor pocket 120 comprises an embossing 121, a top plate 122, at least more than one second coupling hole 123, the temperature sensor insertion hole 124, and the buffer 125.

The embossing 121, which is a part which is in contact with the pet body, has a lower part formed in a round shape and is coupled to the base 110 such that the embossing 121 is exposed to the outside from the inside of the base 110.

In this case, as illustrated in FIG. 4, space is formed between the embossing 121 and the top plate 122 by being recessed inward in a horizontal direction. The space is a part into which the base 110 is fitted when the embossing 121 is coupled to the first coupling hole 111 of the base 110, and functions to securely maintain the coupling of the base 110 to the temperature sensor pocket 120.

The top plate 122 is a part in contact with the bottom surface of the base 110 and is formed to be seated inside the guide 113 of the base 110.

The second coupling hole 123 comprises at least more than one second coupling hole formed through a position of the top plate 122 corresponding to the first protruding pin 112 of the base 110, and is a part which is fitted over and coupled to the first protruding pin 112 of the base 110.

The temperature sensor insertion hole 124 is formed by digging deep into the embossing 121, and the temperature sensor 170 is inserted into the temperature sensor insertion hole 124. That is, this allows the temperature sensor 170 to receive and measure the pet body temperature in a heat transfer method without directly touching the pet skin.

The buffer 125 is formed in the lower part of the center of the top plate 122 by being depressed into the top plate 122 for buffering between the embossing 121 and the temperature sensor 170.

That is, the buffer 125 is formed by being depressed lower than the height of the top plate 122 and functions to prevent the end part of the FPCB 180 coupled to the temperature sensor 170 from protruding to the outside of the top plate 122.

In addition, the buffer 125 has a circular grooved edge 125a formed on the outside thereof and is formed to be inclined gradually toward the circular grooved edge 125a from the temperature sensor insertion hole 124 so that the embossing 121 can maintain elasticity and strength.

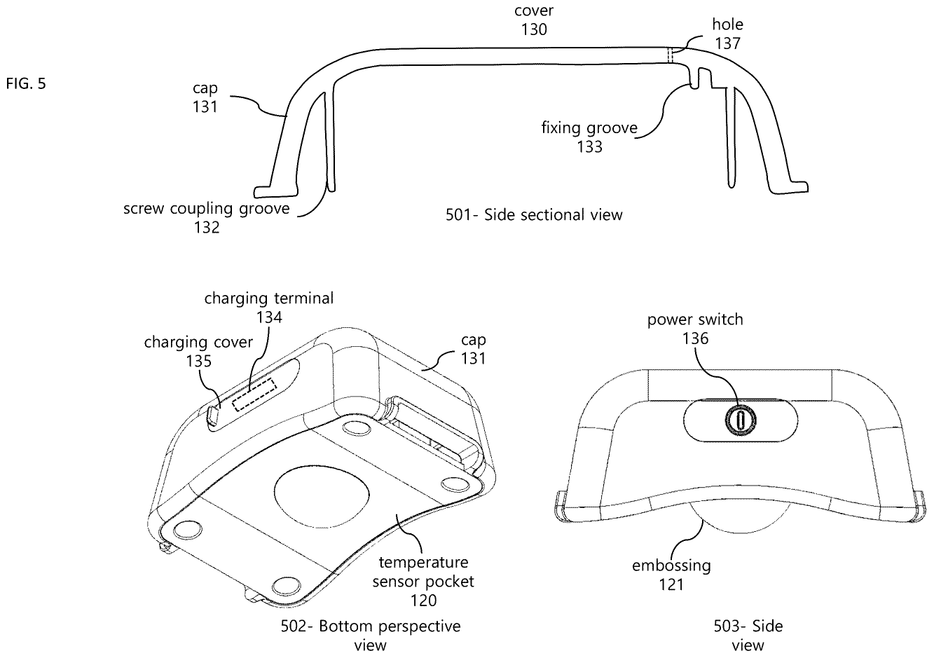

FIG. 5 illustrates a side sectional view 501, a bottom perspective view 502, and a side view 503 respectively illustrating in detail the structure of the cover of the wearable sensor apparatus according to the embodiment of the present disclosure.

As illustrated in FIG. 5, the cover 130 includes the cap 131, at least more than one screw coupling groove 132, at least more than one fixing groove 133, the charging terminal 134, a charging cover 135, a power switch 136, and at least more than one hole 137.

As illustrated in side sectional view 501 of FIG. 5, the cap 131 has space defined therein such that the printed circuit board comprising the battery charging module 140, the battery 150, and the communication and processor module 160 is installed in the space.

At least more than one screw coupling groove 132 is formed in the lower part of the cap 131. In this case, the screw coupling groove 132 is formed at a position corresponding to the screw coupling hole 114 of the base 110, and from the lower part of the base 110, a screw passes through the screw coupling hole 114 and is fastened to the screw coupling groove 132 so that the cap 131 is securely fixed and coupled to the base 110.

The fixing groove 133 is a part for fixing the battery charging module 140, the battery 150, and the communication and processor module 160 inside the cap 131 and is formed at a position corresponding to the fixing hole 141 illustrated in FIG. 2.

As illustrated in bottom perspective view 502 of FIG. 5, the charging terminal 134 is formed on one side of the cap 131 and is a part connected to the charging cable for charging the battery.

The charging cover 135 is provided outside the charging terminal 134 and prevents the charging terminal 134 from being exposed to the outside.

In this case, a handle is formed on an outer first side of the charging cover 135, and a second side of the charging cover 135 is formed to be fixed. Therefore, when charging the battery 150, a user grips and opens the handle and then connects the charging cable to the charging terminal 134.

As illustrated in side view 503 FIG. 5 of, the power switch 136 is formed at a side opposite to the side of the cap 131 in which the charging terminal 134 and the charging cover 135 are formed, and performs the function of turning on or off the power of the wearable sensor apparatus 100.

At least more than one hole 137 is formed through the upper part of the cap 131 and functions to pass light emitted from an LED (not shown) provided inside the cap 131 to the outside so as to display the operation state of the wearable sensor apparatus 100.

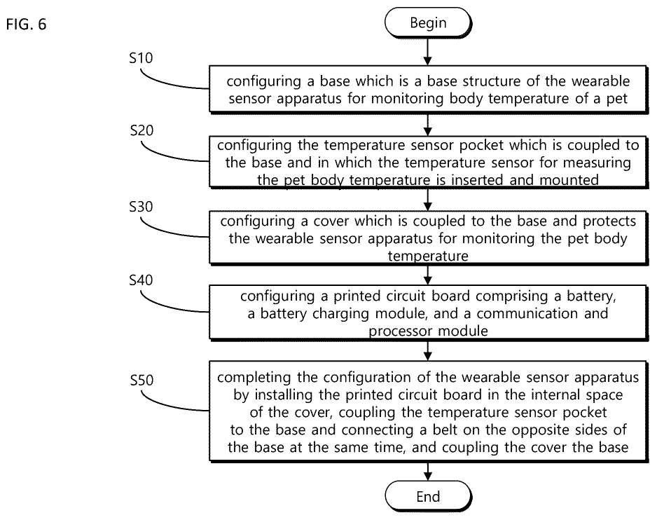

Next, one embodiment of the method of configuring the wearable sensor apparatus, which is configured in this manner, according to the present disclosure will be described in detail with reference to FIG. 6. In this case, the order of each step according to the method of the present disclosure is changed by use environment or a person skilled in the art.

FIG. 6 is a flowchart illustrating in detail the process of configuring the wearable sensor apparatus according to the embodiment of the present disclosure.

As illustrated in FIG. 6, a system (not shown) which produces the wearable sensor apparatus first performs configuring the base 110 which is a base structure of the wearable sensor apparatus 100 at S10.

In addition, it is performed to configure the temperature sensor pocket 120 which is coupled to the base 110 and in which the temperature sensor 170 for measuring the pet body temperature is inserted and mounted at S20.

Particularly, according to the present disclosure, when configuring the temperature sensor pocket 120, it is necessary that the temperature sensor pocket 120 is configured to protrude downward from the base such that the pet body temperature is transmitted to the temperature sensor 170 while the temperature sensor 170 does not directly touch the pet skin such that the temperature sensor 170 can be inserted and mounted inside the temperature sensor pocket 120 configured to protrude downward.

In addition, it is performed to configure the cover 130 which is coupled to the base 110 and protects the wearable sensor apparatus 100 for monitoring the pet body temperature at S30.

In addition, it is performed to configure the printed circuit board comprising the battery 150, the battery charging module 140 which charges the battery, and the communication and processor module 160 in which the pet body temperature measured through the temperature sensor 170 is transmitted to the user portable terminal through short range wireless communication (e.g., Bluetooth), and the result of the pet body temperature measured by the temperature sensor 170 is corrected at S40.

Next, after the printed circuit board configured at S40 is installed in the internal space of the cover 130 configured at S30, the opposite sides of the base 110 are connected to the belt 200 at the same time at which the temperature sensor pocket 120 configured at S20 is coupled to the base 110 configured at S10. In addition, the cover 130 is coupled to the base 110 to complete the configuration of the wearable sensor apparatus 100 at S50.

Meanwhile, the detailed structure of the base 110, the temperature sensor pocket 120, and the cover 130 configured through S10 to S30 is the same as the structure of the same described in FIG. 3, FIG. 4, and FIG. 5, and thus detailed description thereof will be omitted.

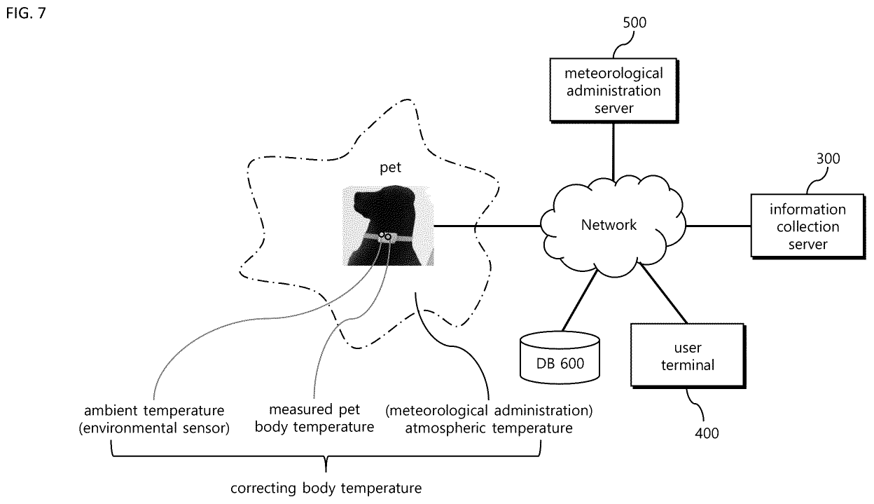

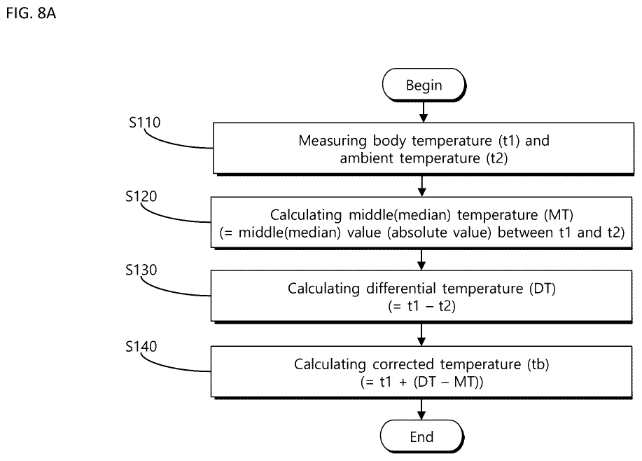

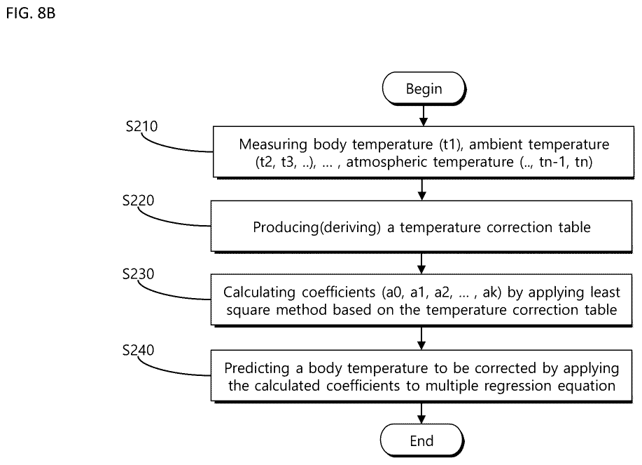

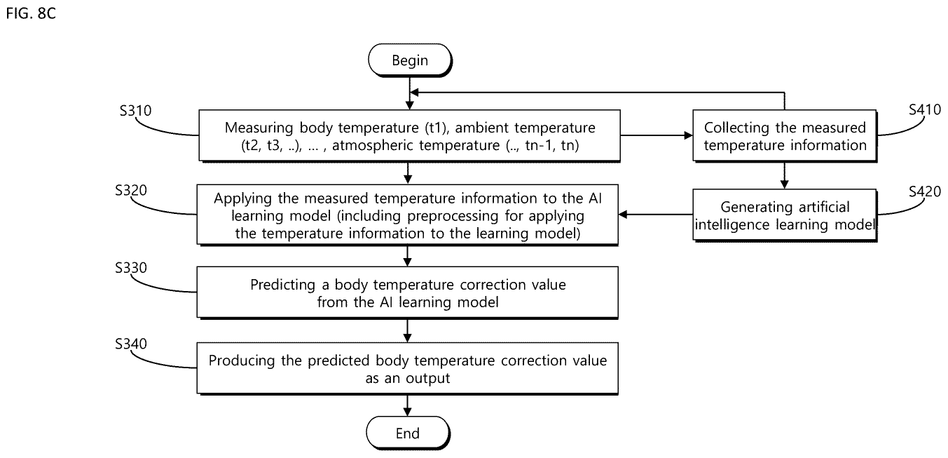

Next, the correction of the value of the pet body temperature measured in the wearable sensor apparatus, which is configured in this manner, according to the present disclosure will be described in detail with reference to FIGS. 7, 8A, 8B, and 8C.

FIG. 7 is a view illustrating the concept of correcting the measured value of the pet body temperature in the wearable sensor apparatus according to the present disclosure, and FIG. 8A to FIG. 8C are flowcharts illustrating the process of correcting the measured value of the pet body temperature for each embodiment in the wearable sensor apparatus according to the present disclosure.

As illustrated in FIG. 7, the wearable sensor apparatus 100 corrects the measured value of the pet body temperature with reference to the pet ambient temperature measured through the environmental sensor 161 and the atmospheric temperature provided from the meteorological administration server 500, and provides the corrected pet body temperature information to the user terminal 400.

In this case, the corrected pet body temperature information is provided to the user terminal 400 via an information collection server 300 through the network, and is stored and managed in a database 600.

In the wearable sensor apparatus 100, the pet body temperature is corrected by applying three methods, and the specific method of the correction is described as follows.

First, the wearable sensor apparatus 100 corrects the pet body temperature measured through the temperature sensor 170 by reflecting the pet ambient temperature measured through the environmental sensor 161 in the pet body temperature, and outputs the correction value of the pet body temperature to the user terminal 400.

That is, a pet body temperature is measured through the temperature sensor installed in the temperature sensor pocket which allows the temperature sensor not to directly touch the pet skin and is configured such that the pet body temperature is transmitted to the temperature sensor, and the ambient temperature is measured through the at least more than one environmental sensor which measures the ambient temperature, and then the pet body temperature measured by the temperature sensor and the ambient temperature measured by the at least more than one environmental sensor are added as independent variables to correct the pet body temperature.

More specifically, a corrected temperature is obtained by using temperature difference DT between the pet body temperature and the ambient temperature, and a middle temperature MT. In this case, the corrected temperature allows the pet body temperature to be corrected according to the ambient temperature, and the pet body temperature is corrected according to the ambient temperature to be a stable measured value. The ambient temperature is a relatively accurate value and has little effect on the pet body temperature, but the pet real body temperature is influenced by the ambient temperature and is out of the range of the body temperature, and thus the pet body temperature is calculated as a value corrected by applying the ambient temperature to the pet body temperature, so the pet body temperature is accurately measured.

To describe in more detail with reference to FIG. 8A, the wearable sensor apparatus 100 measures the measured value t1 of the pet body temperature and the ambient temperature t2 of a pet through the temperature sensor 170 and the environmental sensor 161, respectively, at S110, and checks the middle temperature MT by calculating a middle value (an absolute value) between the measured value t1 of the body temperature and the ambient temperature t2 of a pet at S120.

Next, the wearable sensor apparatus 100 calculates the temperature difference DT between the measured value t1 of a pet body temperature and the ambient temperature t2 of a pet at S130, and a value obtained by subtracting the middle temperature MT calculated at S120 from the temperature difference DT calculated at S130 is added to the measured value t1 of the pet body temperature measured at S110, so a final corrected temperature tb is calculated at S140. Here, at least more than one ambient temperature is measured to be used. When there is information about a plurality of ambient temperatures, the mean, median, standard deviation, and variance of these ambient temperatures are used.

In addition, the wearable sensor apparatus 100 further corrects the pet body temperature with reference to the pet body temperature measured through the temperature sensor 170, the pet ambient temperature measured through the environmental sensor 161, and the atmospheric temperature provided from the meteorological administration server 500. That is, after performing correction of the pet body temperature once more based on the atmospheric temperature collected from the meteorological administration server 500 in the method of 8A, the correction value of the pet body temperature is output.

To describe more specifically with reference to FIG. 8B, the wearable sensor apparatus 100 checks the measured values of the pet body temperature (e.g., t1) and the pet ambient temperatures (e.g., t2, t3, . . . ) measured respectively through the temperature sensor 170 and the environmental sensor 161, and the atmospheric temperatures (e.g., . . . , tn−1, tn) provided from the meteorological administration server 500 at S210, and based on this, and a temperature correction table is produced (derived) based on the measured values and stored at S220.

Next, at S230, the wearable sensor apparatus 100 calculates coefficients (e.g., a0, a1, a2, . . . , and ak) which minimize errors and are used in the multiple regression equation by using the least square method based on the temperature correction table produced at S220.

Equation 1 expresses the multiple regression equation as a linear function of a plurality of independent variables t11, t12, t1k, . . . , and tnk and an error term e. Here, coefficients a0, a2, . . . , and ak are parameters to be predicted.

$$( \begin{matrix}{tb}_{1} \\ {tb}_{2} \\ {tb}_{3} \\ ⋮ \\ tb \text{?}\end{matrix} ) = ( \begin{matrix}{a}_{0} + {a}_{1} + {t}_{11} + … + {a}_{k} {t}_{1 k} \\ {a}_{0} + {a}_{1} + {t}_{21} + … + {a}_{k} {t}_{2 k} \\ {a}_{0} + {a}_{1} + {t}_{31} + … + {a}_{k} {t}_{3 k} \\ ⋮ \\ {a}_{0} + {a}_{1} + t \text{?} + … + {a}_{k} t \text{?}\end{matrix} ) + ( \begin{matrix}{e}_{1} \\ {e}_{2} \\ {e}_{3} \\ ⋮ \\ e \text{?}\end{matrix} ) \qquad [ Equation 1 ]$$

$$\text{?} \text{indicates text missing or illegible when filed}$$

Equation 1 is rearranged like Equation 2, and when Equation 2 is simplified as a formula, Equation 2 becomes like Equation 3, and an error term E is expressed like Equation 4.

$$( \begin{matrix}{tb}_{1} \\ {tb}_{2} \\ {tb}_{3} \\ ⋮ \\ tb \text{?}\end{matrix} ) = ( \begin{matrix}1 + {t}_{11} + … + {t}_{1 k} \\ 1 + {t}_{21} + … + {t}_{2 k} \\ 1 + {t}_{31} + … + {t}_{3 k} \\ ⋮ \\ 1 + t \text{?} + … + t \text{?}\end{matrix} ) ( \begin{matrix}{a}_{0} \\ {a}_{1} \\ {a}_{2} \\ ⋮ \\ {a}_{k}\end{matrix} ) + ( \begin{matrix}{e}_{1} \\ {e}_{2} \\ {e}_{3} \\ ⋮ \\ e \text{?}\end{matrix} ) \qquad [ Equation 2 ]$$

$$\text{?} \text{indicates text missing or illegible when filed}$$

T_{b}=AT+E [Equation 3]

E=t_{b}−AT [Equation 4]

Here, Tb is a body temperature correction value, a is a coefficient, e is an error, A is a matrix representing a set of coefficients for a0 to ak, T is a matrix representing a set of temperatures for t1 to tk, and E is a matrix representing a set of errors for e0 to en.

Next, the process of minimizing errors by using the least square method is developed as in Equation 5 so as to minimize an error term by using Equation 4.

minE²=min (E^{T} E)=min (T_{b}−AT)^{T}(T_{b}−AT) [Equation 5]

When Equation 5 is partially differentiated and set as 0, a predicted value for a coefficient is calculated as in Equation 6.

$$\frac{\text{?}}{\text{?}} ( {E}^{T} E ) = \frac{\text{?}}{\text{?}} ( T \text{?} T \text{?} - 2 {A}^{T} {T}^{T} T \text{?} + {A}^{T} {T}^{T} TA ) = 0 \qquad [ Equation 6 ]$$

$${T}^{T} TA = {T}^{T} T \text{?}$$

$$\tilde{A} = {( {T}^{T} T )}^{- {1}_{{T}^{T} T} \text{?}}$$

$$\text{?} \text{indicates text missing or illegible when filed}$$

Next, at S240, the wearable sensor apparatus 100 finally predicts the pet body temperature by applying the multiple regression equation and the coefficient calculated at S230.

In this case, the wearable sensor apparatus 100 presets a possible range for the pet body temperature, and then considers the pet body temperature as noise when the pet body temperature is out of the range. The wearable sensor apparatus 100 regards the change of the body temperature as increase or decrease due to the ambient temperature t2 and the atmospheric temperature to when the body temperature changes more rapidly than a preset value, and adjusts a weight for a corresponding parameter. That is, by applying the multiple regression equation, a coefficient which minimizes an error e is calculated, stored in a table to be used, and adaptively predicted in real time to perform temperature correction. In addition, even when the ambient temperature suddenly changes, it needs to be filtered and reflected in the temperature correction

Meanwhile, the wearable sensor apparatus 100 corrects the pet body temperature by using an AI learning model in addition to the method of using the multiple regression equation and the least square method illustrated in FIG. 8B. That is, the wearable sensor apparatus 100 predicts the pet body temperature by learning to minimize errors by applying an artificial intelligence algorithm and corrects the pet body temperature. In this case, artificial intelligence learning is preferably performed in the information collection server 300 in FIG. 7, and coefficient prediction for temperature correction is performed in a user terminal 400 in addition to the wearable sensor apparatus 100.

To describe more specifically with reference to FIG. 8C, the wearable sensor apparatus 100 measures the measured values t1 of the pet body temperature and the pet ambient temperatures (e.g., t2, t3, . . . ) measured respectively through the temperature sensor 170 and the environmental sensor 161, and the atmospheric temperatures (e.g., . . . , tn−1, tn) provided from the meteorological administration server 500 at S310. The measured temperature information is collected at S410 and is used as learning information when generating the AI learning model at S420.

Next, the temperature information measured or checked at S310 is preprocessed to be applied to the learning model at S320.

In addition, the wearable sensor apparatus 100 applies the temperature information preprocessed at S320 to the pre-generated learning model, predicts the body temperature correction value from the learning model at S330, and outputs the predicted body temperature correction value at S340.

Accordingly, according to the present disclosure, the pet body temperature can be accurately measured through the wearable sensor apparatus which does not cause trouble on the pet skin despite the contact of the wearable sensor apparatus with the pet skin and has improved thermal conductivity.

In addition, according to the present disclosure, a user can receive and monitor information about the pet body temperature measured by the wearable sensor apparatus by using his or her portable device, so health of a pet managed by the user can be easily checked, and in the event of an abnormality, immediate countermeasures can be taken.

In addition, the result of measuring the pet body temperature is corrected and transmitted to the server in the cloud, and the server in the cloud provides the result of the measured body temperature to a user communication terminal, so the pet health can be accurately monitored while errors of the measurement of the pet body temperature are prevented as much as possible.

As described above, the present disclosure has been described with reference to the embodiments shown in the drawings, but this is only an example, and those skilled in the art will understand that various modified and equivalent other embodiments are possible therefrom. Accordingly, the technical protection scope of the present disclosure should be judged by the following claims.

What is claimed is:

1. A wearable sensor apparatus for monitoring a pet body temperature, the wearable sensor apparatus comprising:
a base configured to constitute a lower part of the wearable sensor apparatus for monitoring the pet body temperature;
a cover configured to be coupled to the base and protect the wearable sensor apparatus; and
a temperature sensor pocket configured to mount a temperature sensor for measuring the pet body temperature,
wherein the temperature sensor pocket is configured to protrude downward from the base such that the pet body temperature is transmitted to the temperature sensor while the temperature sensor pocket prevents the temperature sensor from directly touching skin of the pet.

2. The wearable sensor apparatus of claim 1, wherein the temperature sensor pocket is configured to be made of a thermally conductive material so that the wearable sensor apparatus does not cause trouble on the pet skin even when the wearable sensor apparatus is in contact with the pet skin and has improved thermal conductivity such that the body temperature is accurately measured.

3. The wearable sensor of claim 1, wherein the temperature sensor pocket includes an embossing configured to be exposed to the outside by being coupled to the base, and improve thermal conductivity of the temperature sensor pocket without causing trouble on the pet skin despite contact of the wearable sensor apparatus with the pet skin such that the pet body temperature is accurately measured.

4. The wearable sensor apparatus of claim 1, wherein the base includes:
a first coupling hole through which the temperature sensor pocket passes to the outside at which the pet skin is located such that the temperature sensor pocket is coupled to the base;
at least one first protruding pin configured to fix the temperature sensor pocket;
a guide configured to hold a position of the temperature sensor pocket part;
at least one screw coupling hole configured to couple and fix the cover to the base; and
at least one second protruding pin configured to fasten the wearable sensor apparatus to a belt.

5. The wearable sensor apparatus of claim 3, wherein the temperature sensor pocket includes:
a top plate configured to be in contact with a bottom surface of the base;
at least one second coupling hole configured to be formed in the top plate and fitted over and connected to the first protruding pin of the base;
a temperature sensor insertion hole configured to be depressed into the embossing such that the temperature sensor is inserted into the temperature sensor insertion hole; and
a buffer configured to be depressed into the top plate for buffering between the embossing and the temperature sensor.

6. The wearable sensor apparatus of claim 5, wherein the buffer is configured to be formed by being depressed lower than the top plate such that an end of an FPCB coupled to the temperature sensor is prevented from protruding to the outside of the top plate, and have a circular grooved edge such that the embossing maintains elasticity and strength.

7. The wearable sensor apparatus of claim 1, further comprising:
a battery charging module configured to charge a rechargeable battery; and
a communication and processor module configured to transmit the pet body temperature measured through the temperature sensor to a user terminal or an information collection server,
wherein the cover comprises:
a cap configured to have a space defined therein such that the battery charging module and the communication and processor module are installed in the space;
at least one screw coupling groove configured to be formed in a lower part of the cap by being located at a position corresponding to the screw coupling hole of the base such that the screw coupling groove is screwed to the base;
at least one fixing groove configured to fix the battery charging module and the communication and processor module inside the cap;
a charging terminal configured to be formed on one side of the cap and to be connected to a charging cable for charging the battery;
a charging cover configured to be provided outside the charging terminal and to be opened during the charging of the battery;
a power switch configured to be formed on one side of the cap and to turn on or off power of the wearable sensor apparatus; and
a hole configured to pass light emitted from an LED provided inside the cap to the outside so as to display an operation state of the wearable sensor apparatus.

8. The wearable sensor apparatus of claim 7, wherein the temperature sensor and the communication and processor module are connected to each other by the FPCB, and the FPCB has a power line and a signal line, and the communication and processor module is provided with an antenna for short range wireless communication and a transceiver for wireless communication.

9. The wearable sensor apparatus of claim 7, further comprising:
at least one environmental sensor configured to measure an ambient temperature,
wherein the pet body temperature measured by the temperature sensor and the ambient temperature measured by the at least one environmental sensor are used as independent variables to correct the pet body temperature, and
the corrected pet body temperature is transmitted directly to a user terminal, is transmitted to the user terminal via the information collection server, or is transmitted to the information collection server through relay of the user terminal and the user terminal receives the corrected pet body temperature from the information collection server, so that a monitor of the user is capable of monitoring the pet body temperature.

10. A method of monitoring a pet body temperature, the method comprising:
measuring the pet body temperature through the temperature sensor installed in the temperature sensor pocket configured to protrude downward from the base such that the pet body temperature is transmitted to the temperature sensor while the temperature sensor does not directly touch the pet skin;
measuring the ambient temperature through the at least one environmental sensor measuring the ambient temperature; and
correcting the pet body temperature by adding the pet body temperature measured by the temperature sensor and an atmospheric temperature measured by at least one environmental sensor as independent variables,
wherein the corrected pet body temperature is transmitted directly to a user terminal, is transmitted to the user terminal via an information collection server, or is transmitted to the information collection server through relay of the user terminal and the user terminal receives the corrected pet body temperature from the information collection server, so that the user terminal is capable of monitoring the pet body temperature.

11. The method of claim 10, wherein the correcting of the pet body temperature includes:
correcting the pet body temperature by adding difference between the temperature difference and a middle temperature to a measured body temperature after calculating the middle temperature and temperature difference by using the measured body temperature and the ambient temperature,
predicting the pet body temperature by applying a least square method to calculate coefficients of a multiple regression equation after deriving a temperature correction table by adding the ambient temperature, the atmospheric temperature, or combination thereof to the measured body temperature; or
predicting the pet body temperature by applying the measured body temperature, the ambient temperature, the atmospheric temperature, or combination thereof as input data to an AI learning model after generating the AI learning model by using the measured value of the body temperature, the ambient temperature, the atmospheric temperature, or a combination thereof.

12. A method of configuring a wearable sensor apparatus for monitoring a pet body temperature, the method comprising:
configuring a base which is a base structure of the wearable sensor apparatus;
configuring a temperature sensor pocket in which a temperature sensor for measuring the pet body temperature is mounted;
configuring a cover which is coupled to the base and protects the wearable sensor apparatus for monitoring the pet body temperature;
configuring a printed circuit board comprising a battery charging module which charges a battery, and a communication and processor module which transmits the pet body temperature measured by the temperature sensor to a user terminal through short range wireless communication and corrects a result of the pet body temperature measured by the temperature sensor; and
completing the wearable sensor apparatus in such a manner that the printed circuit board is installed in an internal space of the configured cover, opposite sides of the base are connected to the belt at the same time at which the configured temperature sensor pocket is coupled to the configured base, and the cover is coupled to the base,
wherein the temperature sensor pocket prevents the temperature sensor from touching directly to the pet skin and is configured to protrude downward from the base such that the pet body temperature is transmitted to the temperature sensor.
